(19) United States
(12) Patent Application Publication
(10) Pub. No.: US 2008/0269909 A1
(43) Pub. Date: Oct. 30, 2008

(54) SPACER-POLYMETHYLMETHACRYLATE BONE CEMENT

(75) Inventors: SEBASTIAN VOGT, ERFURT (DE); HUBERT BUCHNER, REINHEIM (DE); KLAUS-DIETER KUHN, MARBURG-ELNHAUSEN (DE); UDO GOPP, WEHRHEIM (DE); MARC THOMSEN, HEIDELBERG (DE)

Correspondence Address: NORRIS, MCLAUGHLIN & MARCUS, PA, 875 THIRD AVENUE, 18TH FLOOR, NEW YORK, NY, 10022, US

(73) Assignee: HERAEUS KULZER GMBH, HANAU (DE)

(21) Appl. No.: 12/104,612

(22) Filed: Apr. 17, 2008

(30) Foreign Application Priority Data
Apr. 24, 2007 (DE) 10 2007 019 593.3
Apr. 24, 2007 (DE) 10 2007 029 098.7

(51) Int. Cl. A61F 2/28

(52) U.S. Cl. 623/23.62

(57) ABSTRACT

This describes a polymethylmethacrylate bone cement that is characterized in that it contains a hydrolytically-degradable X-ray opaquer with a Mohs hardness equal to or less than 3 and a water solubility at room temperature of less than 4 g per liter. The polymethylmethacrylate bone cement is used as temporary placeholder.

The subject matter of the invention is a spacer polymethylmethacrylate bone cement that is suitable for the production of temporary placeholders for two-stage revision of articular endoprostheses.

Articular endoprostheses currently have a serviceable life of several years, e.g. 10-15 years on average in the case of cemented hip-joint prostheses. However, there are cases, in which the articular endoprostheses become loose undesirably prior to reaching the usual serviceable life. In this regard, a distinction is being made between septic and aseptic loosening. Microbial pathogens have not been detected yet in cases of aseptic loosening. Aseptic loosening may be due to a large variety of causes. Aseptic loosening is often caused by abrasion on the sliding surfaces of the articular endoprostheses. The loosening process in septic loosening is elicited by microbial pathogens. In this regard, a distinction is made between early and late infections depending on the time of manifestation. Septic loosening is a very serious disease for the patient and, in addition, associated with very high costs. It is common to perform a revision in cases of aseptic and septic loosening. In this regard, a distinction is made between the one-stage and the two-stage revision.

In general, a placeholder, a so-called spacer, is used in the two-stage revision. This spacer fills the space of the previously revised endoprosthesis for several weeks until the manifest infection has subsided. This placeholder function is very important in order to effectively prevent shrinking of the muscles during this period of time and attain stabilization of the resection situation. Moreover, articulating spacers maintain the mobility of the afflicted extremities. This allows mobilization of the patients at an early time.

Spacers are usually produced by the surgeon using conventional PMMA bone cements and suitable molds. In the process, one or more antibiotics are admixed to the PMMA bone cement powder prior to spacer production according to which microbial pathogens are detected in biopsies and after obtaining an antibiogram. The antibiotics are selected specifically for the microbial pathogens that are present. This procedure is very advantageous, in particular in the presence of multiply-resistant pathogens or in the case of mixed infections involving different pathogens.

The development of spacers can be traced back to the original work of Hovelius and Josefsson (Hovelius L, Josefsson G (1979), An alternative method for exchange operation of infected arthroplasty. Acta Orthop Scand 50: 93-96). Other early work on spacers was performed by Younger (Younger A S, Duncan C P, Masri B A, McGraw R W (1997). The outcome of two-stage arthroplasty using a custom-made interval spacer to treat the infected hip. J Arthroplasty 12: 615-623), Jones (Jones W A. Wroblewski B M (1989) Salvage of failed total knee arthroplasty: the ‘beefburger’ procedure. J Bone Joint Surg Br. 71: 856-857.), and Cohen (Cohen J C, Hozack W J, Cuckler J M, Booth R E Jr (1988), Two-stage reimplantation of septic total knee arthroplasty. Report of three cases using an antibiotic-PMMA spacer block. J Arthroplasty 3: 369-377).

McPherson contributed the concept to produce spacers from bone cement exclusively and to perform no re-implantation of original parts of the prosthesis (McPherson E J, Lewonowski K, Dorr L D (1995), Techniques in arthroplasty. Use of an articulated PMMA spacer in the infected total knee arthroplasty. J. Arthroplasty 10: 87-89).

The spacers that have been used thus far are problematic in that they show a certain degree of abrasion because of the very hard X-ray opaquer particles, such as zirconium dioxide and barium sulfate, that are present in the underlying PMMA bone cement. Abrasion events are a very critical event, in particular at the gliding surfaces of articulating spacers. There is an ongoing discussion as to whether the abrasion that is produced during the use of spacers may possibly cause aseptic loosening of the revision endoprostheses in the two-stage revision.

Another problem of the spacers used thus far is that the antibiotic particles incorporated into the PMMA bone cement are dissolved therefrom only on the surface thereof by the action of body fluid. In order to have high initial release, it is therefore common to add very large quantities of antibiotics that are not common in normal PMMA bone cements for permanent fixation of total articular endoprostheses. A release of major quantities of antibiotics over a period of time of several days up to a few weeks is desired.

It has been disclosed in DE 2905878 that the release of antibiotics from PMMA bone cements can be increased by adding sodium chloride or other soluble alkali halogenides. As an alternative, it was proposed in U.S. Pat. No. 4,233,287 to incorporate water-soluble amino acids in PMMA cements in order to improve the release of active ingredient. The essential disadvantage of both of these methods is that the use of major quantities of water-soluble alkali halogenides and/or amino acids in PMMA bone cements, exposed to the action of wound secretions and/or blood on the hard bone cement effecting dissolution of these additives, leads to the local production of hypertonic solutions which are non-physiological.

Sencan et al. investigated the adherence of bacteria to PMMA bone cement containing teicoplanin and calcium sulfate (I. Sencan, I. Sahn, T. Tuzuner, D. Ozdemir, M. Yildirim, H. Leblebicioglu: In vitro bacterial adherence to teicoplanin and calcium sulfate-soaked bone cement. J. Chemother. 17 (2005) 174-178.). He detected a release of major quantities of teicoplanin in the aqueous medium in the first three days followed by the release of lesser quantities of teicoplanin for up to 33 days.

The invention is based on the object to develop a polymethylmethacrylate bone cement for the production of temporary placeholders that can, on the one hand, not release major quantities of hard abrasion particles and, on the other hand, exhibits high antibiotic/antibiotics release when exposed to the action of aqueous media, such as wound secretion or blood. The polymethyl-methacrylate bone cement to be developed should be designed such that antibiotics in lower-lying areas of the bone cement can also be dissolved from the cement by exposure to the action of aqueous body fluids.

The object was met according to the invention by a polymethylmethacrylate bone cement that is characterized in that it contains a hydrolytically-degradable X-ray opaquer with a Mohs hardness equal to or less than 3 and a water solubility at room temperature of less than 4 g per liter.

Preferably, the hydrolytically-degradable X-ray opaquer is micro-porous and may contain a pharmaceutical excipient.

It can also contain zirconium dioxide, barium sulfate or tantalum in addition to the hydrolytically-degradable X-ray opaquer.

Preferably, the total quantity of X-ray opaquer is 5-25 wt. %.

Preferably, the quantity of the hydrolytically-degradable X-ray opaquer with a Mohs hardness equal to or less than 3 and a water solubility at room temperature of less than 4 g per liter is 3 to 12 wt. %.

Calcium carbonate, magnesium carbonate, calcium sulfate dihydrate and calcium sulfate hemihydrate are preferable as hydrolytically-degradable X-ray opaquer. Calcium carbonate (calcite) has a Mohs hardness of 3 and therefore is a very soft X-ray opaquer. It is particularly advantageous that calcium carbonate usually contains no crystal water which may possibly undergo a side reaction involving the formation of ethylene glycol during ethylene oxide sterilization, which is common for PMMA bone cements. Calcium carbonate dissolves in the presence of carbon dioxide-saturated aqueous solutions such as are present in the human body, e.g. in blood, by the action of bicarbonate. Calcium sulfate dihydrate has a Mohs hardness of 2 and therefore is a very soft X-ray opaquer. Calcium sulfate dihydrate dissolves slowly in water and is physiologically non-objectionable.

Calcium sulfate can also have a water content that is between that of calcium sulfate dihydrate and anhydrous calcium sulfate. In addition, calcium sulfate may contain small quantities of magnesium sulfate and strontium sulfate. Calcium carbonate can contain small quantities of physiologically non-objectionable strontium salts and magnesium salts such as strontium sulfate, strontium carbonate, and magnesium carbonate.

The invention also relates to the use of the PMMA bone cement described herein as temporary placeholder.

The PMMA bone cement described can also be used for permanent fixation of articular endoprostheses. In principle, the bone cement is suitable for the implantation of common hip, knee, and shoulder joints. In addition, it is feasible to produce from the bone cement according to the invention 2-dimensional implants that can be used in reconstructing bone defects of the cerebral and facial cranium. In addition, it is also feasible, in principle, to use the bone cement for vertebroplasty and kyphoplasty.

The invention is illustrated in more detail by the following examples without limiting the scope of the invention.

### EXAMPLES

Firstly, 9 cement powders are produced by comminution. The composition is shown in the following table. Examples 1-3 serve as a reference in this context.

| | Composition of the cement powder | | | | |
|---|---|---|---|---|---|
| | | Polymethyl- | | | |
| Example | Dibenzoyl | methacrylate-co- | | | Gentamicin sulfate |
| no. | peroxide | methylacrylate | ZrO₂ | CaSO₄ × 2H₂O | (AK600) |
| 1 | 0.4 g | 33.7 g | 5.9 g | — | 1.66 g (equivalent to |
| | | | | | 1.0 g gentamicin base) |
| 2 | 0.4 g | 33.7 g | 5.9 g | — | 3.33 g (equivalent to |
| | | | | | 2.0 g gentamicin base) |
| 3 | 0.4 g | 33.7 g | 5.9 g | — | 6.66 g (equivalent to |
| | | | | | 4.0 g gentamicin base) |
| 4 | 0.4 g | 33.7 g | 4.0 g | 1.9 g | 1.66 g (equivalent to |
| | | | | | 1.0 g gentamicin base) |
| 5 | 0.4 g | 33.7 g | 4.0 g | 1.9 g | 3.33 g (equivalent to |
| | | | | | 2.0 g gentamicin base) |
| 6 | 0.4 g | 33.7 g | 4.0 g | 1.9 g | 6.66 g (equivalent to |
| | | | | | 4.0 g gentamicin base) |
| 7 | 0.4 g | 33.7 g | 2.0 g | 3.9 g | 1.66 g (equivalent to |
| | | | | | 1.0 g gentamicin base) |
| 8 | 0.4 g | 33.7 g | 2.0 g | 3.9 g | 3.33 g (equivalent to |
| | | | | | 2.0 g gentamicin base) |
| 9 | 0.4 g | 33.7 g | 2.0 g | 3.9 g | 6.66 g (equivalent to |
| | | | | | 4.0 g gentamicin base) |

| | Composition of the cement powder | | | | |
|---|---|---|---|---|---|
| | | Polymethylmethacrylate- | | | |
| Example | Dibenzoyl | co- | | | Gentamicin sulfate |
| No. | peroxide | methylacrylate | ZrO₂ | Opaquer | (AK600) |
| 10 | 0.4 g | 33.6 g | 4.0 g | 2.0 g CaCO₃ | 3.33 g (equivalent to |
| | | | | | 2.0 g gentamicin base) |
| 11 | 0.4 g | 33.6 g | 4.0 g | 2.0 g MgCO₃ | 3.33 g (equivalent to |
| | | | | | 2.0 g gentamicin base) |
| 12 | 0.4 g | 33.6 g | 4.0 g | 1.0 g CaSO₄ × 2H₂O + | 3.33 g (equivalent to |
| | | | | 1.0 g CaCO₃ | 2.0 g gentamicin base) |
| 13 | 0.4 g | 33.6 g | 4.0 g | 1.0 g CaSO₄ × 2H₂O + | 3.33 g (equivalent to |
| | | | | 1.0 g MgCO₃ | 2.0 g gentamicin base) |
| 14 | 0.4 g | 33.6 g | 2.0 g | 4.0 g CaCO₃ | 3.33 g (equivalent to |
| | | | | | 2.0 g gentamicin base) |
| 15 | 0.4 g | 33.7 g | 2.0 g | 4.0 g MgCO₃ | 3.33 g (equivalent to |
| | | | | | 2.0 g gentamicin base) |
| 16 | 0.4 g | 33.7 g | 2.0 g | 2.0 g CaSO₄ × 2H₂O + | 3.33 g (equivalent to |
| | | | | 2.0 g CaCO₃ | 2.0 g gentamicin base) |
| 17 | 0.4 g | 33.7 g | 2.0 g | 2.0 g CaSO₄ × 2H₂O + | 3.33 g (equivalent to |
| | | | | 2.0 g MgCO₃ | 2.0 g gentamicin base) |

Subsequently, 40 g cement powder each are mixed with 20 ml methylmethacrylate, in which 1.0 mass-% dimethyl-p-toluidine is dissolved. A paste is thus formed that is then spread into hollow molds where it cures after a few minutes. The cylinder-shaped test bodies thus generated have a height of 1 cm and a diameter of 2.5 cm. Five test bodies are produced for each cement variant. The test bodies are stored separately in 20 ml distilled water each at 37° C. Each day, all of the release medium is removed and the quantity of gentamicin released into the medium is determined. The test bodies are then stored again in 20 ml of fresh distilled water each at 37° C. The gentamicin content of the eluate is determined using a TDX analyzer made by Abott. The mass of gentamicin base released in each case is listed by test body in the following table as a function of the time of storage of the test bodies in the release medium.

| | Gentamicin release | | |
|---|---|---|---|
| | per form body | | |
| | [μg/form body] | | |
| Sample no. | 1 d | 3 d | 5 d |
| 1 | 1,806 | 74 | 45 |
| 2 | 4,568 | 191 | 141 |
| 3 | 14,386 | 1,507 | 888 |
| 4 | 1,979 | 99 | 122 |
| 5 | 4,672 | 370 | 293 |
| 6 | 18,887 | 2,545 | 1,529 |
| 7 | 2,476 | 134 | 75 |
| 8 | 6,073 | 497 | 286 |
| 9 | 22,602 | 2,565 | 1,659 |
| 10 | 4818 | 367 | 325 |
| 11 | 5169 | 420 | 460 |
| 12 | 5294 | 391 | 353 |
| 13 | 6665 | 515 | 598 |
| 14 | 6344 | 836 | 593 |
| 15 | 6877 | 693 | 478 |
| 16 | 5202 | 415 | 442 |
| 17 | 6166 | 391 | 323 |

In addition, the cements of examples 1-9 are used to produce plates and strips are then cut from the plates. The 4-point flexural strength and the modulus of elasticity are then determined on these strips. The results are shown in the following table. Common PMMA bone cements used for fixation of articular endoprostheses should have a flexural strength in the 4-point bending test of ≧50 MPA and a modulus of elasticity of ≧1800 MPA. The results show that the minimum requirements with regard to flexural strength and modulus of elasticity were met by all cements with the exception of the cement of sample number 9. The cement of example 9 is an exception in that its flexural strength is approximately 5 MPA lower. Even this finding is quite acceptable for a spacer PMMA bone cement, since the spacer PMMA bone cement is implanted only temporarily and does not have to possess permanent strength.

4-point bending

| | Flexural | Modulus of |
|---|---|---|
| Sample | strength | elasticity |
| no. | [MPa] | [MPa] |
| 1 | 60.9 | 2516 |
| 2 | 60.8 | 2651 |
| 3 | 55.4 | 2657 |
| 4 | 61.3 | 2,722 |
| 5 | 53.9 | 2,654 |
| 6 | 51.2 | 2,826 |
| 7 | 52.1 | 2,768 |
| 8 | 54.9 | 2,728 |
| 9 | 45.3 | 2,671 |
| 10 | 61.5 | 2686 |
| 11 | 58.9 | 2859 |
| 12 | 61.2 | 2867 |
| 13 | 56.7 | 2773 |
| 14 | 60.7 | 2859 |
| 15 | 55.6 | 2917 |
| 16 | 59.7 | 2923 |
| 17 | 53.8 | 2863 |

In addition, three segments without antibiotic were produced and their flexural strength and bending modulus were determined.

| | | Polymethyl | |
|---|---|---|---|
| Sample | Dibenzoyl- | methacrylate-co- | |
| no. | peroxide | methylacrylate | Opaquer |
| 18 | 0.4 g | 33.6 g | 6.0 g CaCO₃ |
| 19 | 0.4 g | 33.6 g | 6.0 g MgCO₃ |
| 20 | 0.4 g | 33.6 g | 6.0 g CaSO₄x2H₂O |

4-point bending

| | Flexural | Modulus of |
|---|---|---|
| Example | strength | elasticity |
| no. | [MPa] | [MPa] |
| 18 | 58.5 | 2820 |
| 19 | 58.9 | 2702 |
| 20 | 60.0 | 2619 |

Subsequently, spacer PMMA bone cements containing barium sulfate and containing tantalum as additional X-ray opaquer were also produced. Powdered barium sulfate and tantalum dust were used in the process. The cements of examples 21 and 24 were mixed without any problems and exhibited a release of active ingredient that was comparable to the test bodies of example 7.

| | Composition of the cement powder | | | | |
|---|---|---|---|---|---|
| | | Polymethyl- | | | |
| Example | Dibenzoyl- | methacrylate-co- | | Degradable | Gentamicin sulfate |
| no. | peroxide | methylacrylate | Opaquer | opaquer | (AK600) |
| 21 | 0.4 g | 33.7 g | 2.0 g | 3.9 g | 1.66 g (equivalent to |
| | | | barium | CaSO₄ × 2H₂O | 1.0 g gentamicin base) |
| | | | sulfate | | |
| 22 | 0.4 g | 33.7 g | 2.0 g | 3.9 g | 1.66 g (equivalent to |
| | | | tantalum | CaSO₄ × 2H₂O | 1.0 g gentamicin base) |
| | | | powder | | |
| 23 | 0.4 g | 33.7 g | 2.0 g | 3.9 g | 1.66 g (equivalent to |
| | | | barium | CaCO₃ | 1.0 g gentamicin base) |
| | | | sulfate | | |
| 24 | 0.4 g | 33.7 g | 2.0 g | 3.9 g | 1.66 g (equivalent to |
| | | | tantalum | MgCO₃ | 1.0 g gentamicin base) |
| | | | powder | | |

1. A Polymethylmethacrylate bone cement, comprising a hydrolytically-degradable X-ray opaquer having a Mohs hardness equal to or less than 3 and a water solubility at room temperature of less than 4 g per liter.

2. The Polymethylmethacrylate bone cement according to claim 1, wherein the hydrolytically-degradable X-ray opaquer is micro-porous.

3. The Polymethylmethacrylate bone cement according to claim 1, further comprising zirconium dioxide, barium sulfate or tantalum.

4. The Polymethylmethacrylate bone cement according to claim 1, wherein the hydrolytically-degradable x-ray opaquer is selected from the group consisting of calcium carbonate, magnesium carbonate, calcium sulfate dihydrate, calcium sulfate hemihydrate and mixtures thereof.

5. The Polymethylmethacrylate bone cement according to claim 1, wherein the total quantity of X-ray opaquer is 5-25 wt. %.

6. A method for temporarily placeholding in a two-stage revision of articular endoprosthese comprising filling a space of a previously revised endoprosthesis with a polymethylmethacrylate bone cement comprising a hydrolytically-degradable x-ray opaquer having Mohs hardness equal to or less than 3 and a water solubility at room temperature of less than 4 g per liter.

7. A method for permanent fixation of articular endoprostheses comprising implanting a endoprostheses having a polymethylmethacrylate bone cement comprising a hydrolytically-degradable x-ray opaquer having Mohs hardness equal to or less than 3 and a water solubility at room temperature of less than 4 g per liter.

8. The polymethylmethacrylate bone cement according to claim 2, wherein the hydrolytically-degradable x-ray opaquer contains a pharmaceutical excipient.
